(19) United States
(12) Patent Application Publication
(10) Pub. No.: US 2011/0077440 A1
(43) Pub. Date: Mar. 31, 2011

(54) Process for Producing Alkyl Substituted Indanes

(76) Inventors: Adeniyi Lawal, Livingston, NJ (US); Obiefuna C. Okafor, Irvington, NJ (US); Sunitha Rao Tadepalli, Manalapan, NJ (US); Geatesh Karunakaran Tampy, Colts Neck, NJ (US)

(21) Appl. No.: 12/568,268

(22) Filed: Sep. 28, 2009

(51) Int. Cl. C07C 13/465

(52) U.S. Cl. 585/360

(57) ABSTRACT

The present invention relates to an improved process for producing alkyl substituted indanes which are used in the synthesis of fragrance ingredients for perfumery applications.

### FIELD OF INVENTION

The present invention relates to an improved process for producing alkyl substituted indanes which are used in the synthesis of fragrance ingredients for perfumery applications.

### BACKGROUND OF INVENTION

Microreactor technology is an emerging field that has recently sparked intensive efforts among both academic and industrial participants.

U.S. Pat. No. 2,851,501, U.S. Pat. No. 4,440,966 and British Pat. No. 991146 describe a method for the production of indanes by reacting styrene and an alkene in the presence of an acid catalyst in a batch or semi-batch reactor. In these systems, the reaction medium usually needs to be stirred vigorously to achieve the formation of a meta-stable-emulsion that is required for high yield of the indanes. Such intense mixing requires huge energy requirements which may be difficult to achieve in the production scale batch reactors.

For such fast reactions, a different approach can be used to make the process more energy efficient. Microchannel reactors, by virtue of their small (sub-millimeter) transverse dimensions, possess extremely high surface to volume ratios and consequently, exhibit enhanced heat and mass transfer rates. Heat and mass transfer coefficients that are at least one order of magnitude higher than obtained in conventional reactors have been reported in microchannel reactors. The enhanced mass transfer rates in the microreactor enable the realization of fast, close to intrinsic kinetics, with improved reactor productivity and/or superior product conversion and selectivity in less time when compared to conventional batch process. The use of microchannel reactor technology for various liquid phase reactions have shown improved product yield by either taking advantage of enhanced heat transfer or mass transfer rates.

The present invention uses these advantages of microreactors for the synthesis of a mixture of alkyl substituted indane isomers known as PMI constituting primarily of 3 isomers: penta-methyl indane, ethyl tri-methyl indane and ethyl tetra-methyl indane. PMI is an important intermediate in the production of valuable fragrance ingredients.

### SUMMARY OF THE INVENTION

The present invention provides a process for the production of alkyl substituted indane isomers, wherein the process comprises of reacting a mixture of isoamylenes and alpha-methyl styrene in the presence of a catalyst in a microchannel reactor system and producing the alkyl substituted indane isomers.

### BRIEF DESCRIPTION OF THE DRAWINGS

FIG. 1: This figure illustrates the microreactor setup for the production of alkyl substituted indane isomers.

### DETAILED DESCRIPTION OF THE INVENTION

According to the present invention, the process for the production of alkyl substituted indane isomers comprises of reacting a mixture of iso-amylenes and alpha-methyl styrene in the presence of a catalyst in a microchannel reactor system and producing the alkyl substituted indane isomers.

According to the present invention, the alkyl substituted indane isomers is selected from the group comprising: penta-methyl indane, ethyl tri-methyl indane and ethyl tetra-methyl indane.

As used herein, the phrase “reactor” refers to the device where the reaction actually occurs. As used herein, the term “microreactor” and “microchannel reactor” refers to a device or an assemblage of related devices that contains reaction channels in which at least one of the transverse dimensions is sub-millimeter.

In some embodiments of the present invention, the reactor may be a packed bed reactor, wherein the reactor is packed with materials such as glass beads, catalyst (about 10 μm to about 100 μm particle size). As used herein, the terms “packed” and “packing” mean to fill with an amount of material that allow effective production of a pre-determined amount of alkyl substituted indanes and the amount of material often requires taking into consideration, e.g., the size of the reactor vessel, the material type and the predetermined amount of alkyl substituted indanes.

In some embodiments of the present invention, the catalyst for producing the alkyl substituted indane isomers can be a mineral acid such as sulfuric acid, phosphoric acid etc. or organic acid such as para toluene sulfonic acid. In such embodiments, the acid strength of the catalyst may vary from 55 to 95 wt %, the catalyst to reactant mole ratio may vary from about 0.1 to about 5, and iso-amylene to alpha methyl styrene mole ratio may vary from 0.5 to about 2. In such embodiments, the reaction temperature may range from 0 to 100° C. and the reaction may be carried out at low pressure i.e. atmospheric pressure (14.7 psig) to 100 psig.

In some embodiments of the present invention, the catalyst for producing the alkyl substituted indane isomers can be a solid catalyst such as, but not limited to, solid acid catalysts available commercially such as Filtrol-24, Filtrol-13, and Amberlyst. In some embodiments, the solid acid catalyst can also be prepared in-house using various techniques such as wet impregnation, sol-gel etc. In such embodiments, the acid content of the solid acid catalyst may vary from about 0.1 to about 5 wt % with the support constituting the remaining weight of the catalyst. The mole ratio of iso-amylene to alpha methyl styrene may vary from about 0.5 to about 2. In such embodiments, the reaction temperature may vary from 30 to about 300° C. and the reaction pressure may vary from atmospheric pressure (14.7 psi or 0 psig) to 1000 psig.

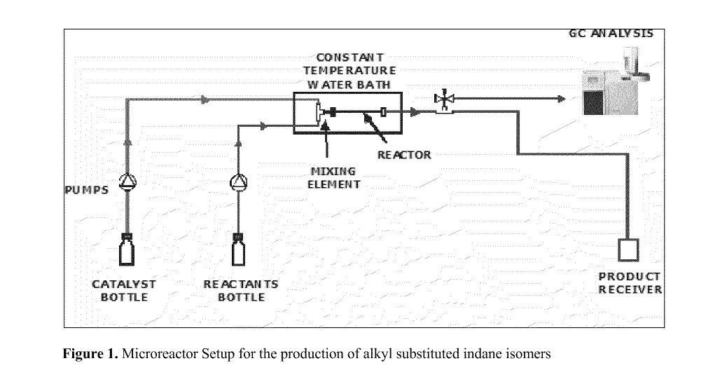

In some embodiments of the present invention, the process for the production of alkyl substituted indane isomers is illustrated by, but not limited to, the following experimental procedure and FIG. 1. The reactants alpha methyl styrene and iso-amylenes were premixed and pumped to the reactor. In some embodiments, the catalyst was also pumped to the reactor. In some embodiments, both the reactants and the catalyst were contacted in a tee (i.e., a T-junction, which is a point where one means of delivery meets another without crossing it, thus, forming a “T” between them) before entering the reactor. In some embodiments, the reactor comprised of channels with internal diameter (ID) ranging from 0.5 to 3 mm. In some embodiments, the reactor was packed with inert glass beads or catalyst and placed in a heating circulating water bath. From the reactor, the reaction mixture was collected in a product receiver. In some embodiments, the microreactor setup may comprise a back pressure regulator after the reactor to obtain the desired pressure for the reaction. The organic layer of the reaction mixture was extracted, neutralized and washed to remove any traces of acid and analyzed using Gas Chromatography (GC).

In some embodiments, the process of the present invention provides a high-energy efficiency, wherein the energy efficiency is measured from the rate of heat removed from the reactor during the reaction. As used herein, “high-energy efficiency” refers to a high heat removal rate per volume of reactor in the microreactor process compared to batch process.

The following are provided as specific embodiments of the present invention. Other modifications of this invention will be readily apparent to those skilled in the art. Such modifications are understood to be within the scope of this invention. All reagents except iso-amylenes and the solid acid catalysts were obtained from Sigma Aldrich. Iso-amylenes were obtained as commercial grade from IFF production plants. As used herein all percentages are weight percent unless otherwise noted, L is understood to be liter, mL is understood to be milliliter, psig is understood to be pounds per square inch guage, g is understood to be gram, min is understood to be minutes and hr to be hour. Productivity of PMI expressed as the Space-Time Yield or average reaction rate (ARR) and the yield calculated as,

$$ARR = \frac{{x}_{prod} \cdot {F}_{reactants}}{ɛ {V}_{MR}}$$

$$Yield = \frac{Amount of PMI formed}{Total amount of reactants fed in the reactor}$$

Residence time for experiments with liquid catalyst is calculated as,

$$Residence time = \frac{ɛ {V}_{MR}}{{F}_{reactants} + {F}_{catalyst}}$$

Residence time for experiments with solid catalyst is calculated as,

$$Residence time = \frac{ɛ {V}_{MR}}{{F}_{reactants}}$$

Energy removal rate is calculated as,

$$Energy removal rate = \frac{Heat of reaction . ARR .1000}{Molecular weight of product . 3600}$$

where X is weight fraction, F_{reactants} is mass flow rate of reactants into the microreactor (g/hr), ε is the fractional void space of the packed bed, V_{MR} is the volume of the microreactor (L), Heat of reaction (kJ/mole) and Molecular weight of product (g/mole). IFF as used herein is understood to mean International Flavors & Fragrances Inc., New York, N.Y., USA.

### Example 1

### Production of PMI Using 72 Wt % Recycled Sulfuric Acid Catalyst in a Microchannel Reactor

A mixture of isoamylenes (15 wt % 2-methyl-1-butene and 85 wt % 2-methyl-2-butene) and alpha-methyl styrene at a molar ratio of 1.44 is fed at flow rate of 0.1 mL/min into a 91 cm microchannel reactor with an internal diameter of 2.4 mm. A catalyst stream of 72 wt % recycled sulfuric acid is also fed simultaneously at 0.1 mL/min to the microchannel reactor via a t-shaped mixing element. The reactor is packed with inert glass beads of a size range of 75 to 150 μm, kept at a constant temperature of 38° C. and the system pressure is 0 psig. The product mixture containing organic and acid layers are separated and a sample of the organic layer is analyzed by GC. The yield of PMI is 0.15 g per g reactants. The Space-Time Yield or average reaction rate for the product is 415 g/L.hr, with a residence time of 496 seconds (8 minutes) in the microchannel reactor. The energy or heat removal rate in the microchannel reactor under these conditions is 495 kW/m³.

### Comparative Example Using Lab-Scale Semi-Batch Reactor

600 gm of 72 wt % recycled sulfuric acid is introduced into a 2 L semi-batch reactor at the beginning of the reaction. The reactor is kept at a constant temperature of 38° C. and is run under autogenous pressure at an agitation rate of 275 rpm. A mixture of isoamylenes (15 wt % 2-methyl-1-butene and 85 wt % 2-methyl-2-butene) and alpha-methyl styrene at a molar ratio of 1 is fed at flow rate of 3.5 g/min into the reactor for 5 hours and then aged for 30 minutes. The product mixture containing organic and acid layers are separated and a sample of the organic layer is analyzed by GC. The yield of PMI is 0.42 g per g reactants. The Space-Time Yield or average reaction rate for the product is 49.2 g/L.hr, with a residence time of 5 hours in the reactor. The energy or heat removal rate in the semi-batch reactor under these conditions is 17.6 kW/m³.

Comparative Example Using Production-Scale Continuous Reactor

2000 kg of 72 wt % recycled sulfuric acid is introduced into a 12 m³ CSTR at the beginning of the reaction. The reactor is kept at a constant temperature of 35° C. and is run under autogenous pressure at a power to volume ratio of 1.3 kW/m³. A mixture of isoamylenes (15 wt % 2-methyl-1-butene and 85 wt % 2-methyl-2-butene) and alpha-methyl styrene at a molar ratio of 1:0.84 is fed at flow rate of 1000 kg/hr into the reactor for 9 hours and then product mix is continuously removed from the reactor and the organic layer is separated from the acid layer. The yield of PMI is 0.62 g per g reactants. The Space-Time Yield or average reaction rate for the product is 50 g/L.hr, with a residence time of 9 hours in the reactor. The energy or heat removal rate in this reactor under these conditions is 15.2 kW/m³.

### Example 2

### Production of PMI Using 90 Wt % Sulfuric Acid Catalyst in a Microchannel Reactor

A mixture of isoamylenes (15 wt % 2-methyl-1-butene and 85 wt % 2-methyl-2-butene) and alpha-methyl styrene at a molar ratio of 1 is fed at flow rate of 1.5 mL/min into a 6 cm microchannel reactor with an internal diameter of 2.4 mm. A catalyst stream of 90 wt % sulfuric acid is also fed simultaneously at 1.5 mL/min to the microchannel reactor via a t-shaped mixing element. The reactor is packed with inert glass beads of a size range of 75 to 150 μm, kept at a constant temperature of 95° C. and the system pressure is 0 psig. The product mixture containing organic and acid layers are separated and a sample of the organic layer is analyzed by GC. The yield of PMI is 0.643 g per g reactants. The Space-Time Yield or average reaction rate for the product is 424,810 g/L.hr, with a residence time of 2.2 seconds in the microchannel reactor. The energy or heat removal rate in the microreactor under these conditions is 127,832 kW/m³.

### Comparative Example Using Semi-Batch Reactor

4 mL of 90 wt % sulfuric acid is introduced into a 25 mL semi-batch reactor at the beginning of the reaction. The reactor is kept at a constant temperature of 38° C. and is run under autogenous pressure with an agitation rate of 1,500 rpm. A mixture of isoamylenes (15 wt % 2-methyl-1-butene and 85 wt % 2-methyl-2-butene) and alpha-methyl styrene at a molar ratio of 1 is fed at flow rate of 0.08 mL/min into the reactor for 3 hours and then aged for 1 hour. The yield of PMI is 0.083 g per g reactants. The Space-Time Yield or average reaction rate for the product is 14.3 g/L.hr, with a residence time of 4 hours in the reactor. The energy or heat removal rate in the semi-batch reactor under these conditions is 30 kW/m³.

### Example 3

### Production of PMI Using Filtrol-24 Solid Catalyst in a Microchannel Reactor

A mixture of isoamylenes (15 wt % 2-methyl-1-butene and 85 wt % 2-methyl-2-butene) and alpha-methyl styrene at a molar ratio of 1 is fed at flow rate of 0.05 mL/min into a 6 cm microchannel reactor with an internal diameter of 2.4 mm. The reactor is packed with Filtrol-24 (a solid acid catalyst) with particle size range of 75 to 150 μm, kept at a constant temperature of 140° C. and the system pressure is 300 psig. The yield of PMI is 0.46 g per g reactants. The Space-Time Yield or average reaction rate for the product is 6 g/g catalyst .hr, with a residence time of 130 seconds in the microchannel reactor. The energy or heat removal rate in the microreactor under these conditions is 4645 kW/m³.

What is claimed:

1. A process for the production of alkyl substituted indane isomers, the process comprising of reacting a mixture of isoamylenes and alpha-methyl styrene in the presence of a catalyst in a microchannel reactor system and producing the alkyl substituted indane isomers.

2. The process of claim 1 wherein the alkyl substituted indane isomers is selected from the group comprising: penta-methyl indane, ethyl tri-methyl indane and ethyl tetra-methyl indane.

3. The process of claim 1 wherein the reactor system is a microreactor.

4. The process of claim 1 wherein the reactor diameter can vary from 0.5 to 3 mm.

5. The process of claim 1 wherein the reactor system is a packed bed reactor comprising of packing material which can be glass beads, solid acid catalysts or mixture of the two.

6. The packing material of claim 5 can have particle size ranging from about 10 μm to about 100 μm.

7. The process of claim 1 wherein the mole ratio of iso-amylene to alpha methyl styrene is from about 0.5 to about 2.

8. The process of claim 1 wherein the catalyst is a liquid acid.

9. The process of claim 8 wherein the acid strength may vary from 55 to 95 wt %.

10. The process of claim 8 wherein the catalyst to reactant mole ratio may vary from about 0.1 to about 5.

11. The process of claim 8 wherein the reaction temperature is from about 0° C. to about 100° C.

12. The process of claim 8 wherein the reaction is carried out under a pressure from about 0 psig to about 100 psig.

13. The process of claim 1 wherein the catalyst is a solid acid catalyst.

14. The process of claim 13 wherein the acid content of the solid acid catalyst may vary from about 0.1 to about 5 wt %.

15. The process of claim 13 wherein the reaction is carried out in the temperature ranging from 30 to about 300° C.

16. The process of claim 13 wherein the reaction is carried out in the pressure range from about atmospheric (0 psig) to about 1000 psig.
